(19) United States
(12) Patent Application Publication
(10) Pub. No.: US 2012/0186329 A1
(43) Pub. Date: Jul. 26, 2012

(54) PARTICLE SENSOR, EXHAUST SYSTEM AND METHOD FOR DETERMINING PARTICLES IN THE EXHAUST GAS

(75) Inventors: Yasser M. Yacoub, Koln (DE); Guido Havenith, Herzogenrath (DE)

(73) Assignee: FORD GLOBAL TECHNOLOGIES, LLC, Dearborn, MI (US)

(21) Appl. No.: 13/351,635

(22) Filed: Jan. 17, 2012

(30) Foreign Application Priority Data
Jan. 20, 2011 (DE) 102011002937.0

(51) Int. Cl. G01N 33/497

(52) U.S. Cl. 73/23.31

(57) ABSTRACT

A particle sensor for exhaust system is provided. In one embodiment, the particle sensor comprises a sensor element and an integrated amplifier circuit for amplifying a reference voltage for the sensor element. In this way, the particle sensor may provide increased sensitivity.

### RELATED APPLICATIONS

The present application claims priority to German Patent Application No. 102011002937.0, filed on Jan. 20, 2011, the entire contents of which are hereby incorporated by reference for all purposes.

### FIELD

The disclosure relates to a particle sensor, an exhaust system and a method for determining particles in the exhaust gas.

### BACKGROUND AND SUMMARY

To monitor an exhaust system of an internal combustion engine, use is made of particle sensors which measure the particle content of the exhaust gases flowing through the exhaust system.

At present, resistive particle sensors are known in which two or more metallic electrodes are formed. The particles, in particular soot particles, that are accumulated cause the electrodes, which engage into one another in a comb-like manner, to be short-circuited, and therefore, with increasing particle concentration on the sensor surface, a decreasing resistance or a decreasing impedance (or an increasing current if a constant voltage is applied) can be measured between the electrodes. The measured current or the change thereof can be correlated with the accumulated mass of particles and therefore also with the particle concentration prevailing in the exhaust gas.

Sensors or systems of said type are known for example from DE102008041809A1 and EP1873511A2/A3.

The sensitivity of the specific resistance of a particle sensor with respect to the accumulated particles may be increased by raising the reference voltage that is applied to the sensor element of the particle sensor. The reference or supply voltage for such a sensor element is typically set at 12 volts or 5 volts, such as is available from the 12 volt battery system. This is the case in particular for applications of lightweight construction.

An increase of the supply voltage to a higher level, for example to 30-40 volts, requires either an increase in the voltage of the battery supply to a higher voltage, for example 42 volts, or an amplification of the supply voltage, proceeding from 12 volts, to the required sensor voltage, which takes place in a control unit of the drivetrain PCM (power train control module).

The first approach entails an increase in costs which is not justified merely by a certain requirement of measurement, wherein the second approach may be prohibitive because it requires an amplification to the required voltage in the control unit of the drivetrain, and cabling to the position of the sensor in the exhaust-gas flow. This is not desirable on account of influences caused by electromagnetic force (EMF) in an environment which operates primarily with a 12 volt supply and, in most cases, a 5 volt supply in the region of the sensors/actuators.

The inventors herein have recognized the issues with the above approaches and offer a sensor to at least partly address them. In one embodiment, a particle sensor for an exhaust system comprises a sensor element and an integrated amplifier circuit for amplifying a reference voltage for the sensor element.

In this way, the original voltage supplied by a battery, for example, may be amplified by the amplifier circuit. In doing so, the sensitivity of the particle sensor may be increased without the EMF interference associated with amplifying the voltage in the PCM.

The above advantages and other advantages, and features of the present description will be readily apparent from the following Detailed Description when taken alone or in connection with the accompanying drawings.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows a schematic illustration of the interconnection of a particle sensor according to an embodiment of the disclosure.

FIG. 2 shows a first variant of an amplifier circuit of the particle sensor according to an embodiment of the disclosure.

FIG. 3 shows a second variant of an amplifier circuit of the particle sensor according to an embodiment of the disclosure.

FIG. 4 shows a measurement circuit of the particle sensor according to an embodiment of the disclosure.

FIG. 5 shows a flow chart illustrating an example method for a particle sensor according to an embodiment of the present disclosure.

### DETAILED DESCRIPTION

According to a first aspect of the disclosure, a particle sensor for an exhaust system comprises a sensor element and an integrated amplifier circuit for amplifying a reference voltage for the sensor element.

In general, the particle or solid body sensor may be used in the exhaust system to detect solid and also soluble fractions in the exhaust-gas flow. For this purpose, use is conventionally made of a resistance element whose resistance varies when substances from the exhaust gas precipitate on the sensor element. This requires regular regeneration of the sensor by periodically increasing the temperature of the sensor element in order to evaporate the accumulated material. The derivative of the sensor signal with respect to time may be used to calculate the mass throughflow of the solid or soluble materials in the exhaust gas. Such a sensor concept may also be considered to be a means for the detection of a leak of a particle filter if soot emissions downstream of the filter exceed a diagnostic threshold.

The proposed particle sensor uses a local amplification of the supply voltage at the position of the sensor. Flexibility and design freedom are gained in this way, because the desired reference voltage can be attained independently of the control unit of the drivetrain and the manufacturer thereof, and because a minimization of adverse effects caused by the EMF is realized. Furthermore, the particle sensor may be used with any existing system because externally standardized terminals and voltages are used.

The amplifier circuit may be arranged in a plug connector of the particle sensor. The plug connector may be a plug connector for connecting to a wiring loom of the motor vehicle. The arrangement of the amplifier circuit in the plug connector permits an amplification of the reference voltage at the input of the particle sensor, that is to say even before the sensor element, and thus permits a simple internal construction of the particle sensor.

The amplifier circuit can generate a reference voltage of 30-40 volts. Proceeding from a conventional supply or reference voltage of 12 volts, the amplifier circuit amplifies the reference voltage to an elevated voltage level of approximately 30-40 volts, which makes an increase in measurement sensitivity possible.

The amplifier circuit may have a direct-current converter. The direct-current converter can provide an amplified reference voltage in a simple manner. For example, a direct-current converter may be used to generate a reference voltage of 30 volts.

The amplifier circuit may have a voltage regulator. A voltage regulator may provide a certain voltage, for example 10 volts, from the 12 volt supply. A required or defined amplified reference voltage can be precisely set by means of a combination of several or individual voltage regulators and/or direct-current converters.

The particle sensor may comprise a measurement circuit connected to the sensor element. The measurement circuit evaluates the sensor element, for example by direct or indirect measurement of the resistance, and can output a measurement signal or sampled signal.

The amplifier circuit may be connected to the measurement circuit in order to provide the amplified reference voltage. The amplifier circuit can thus provide the reference voltage and/or supply voltage for the measurement circuit.

According to a second aspect of the disclosure, an exhaust system for a motor vehicle with an internal combustion engine comprises a particle sensor as described above. The same advantages and modifications as those described above apply.

According to a further aspect of the disclosure, a method for determining particles in the exhaust gas of an internal combustion engine with a particle sensor comprises providing a reference voltage; amplifying the reference voltage in the particle sensor; and determining particles by means of the particle sensor.

The same advantages and modifications as those described above apply.

The drawings serve merely for the explanation of the disclosure, and do not restrict the disclosure. The drawings and the individual parts are not necessarily drawn to scale. The same reference symbols are used to denote identical or similar parts.

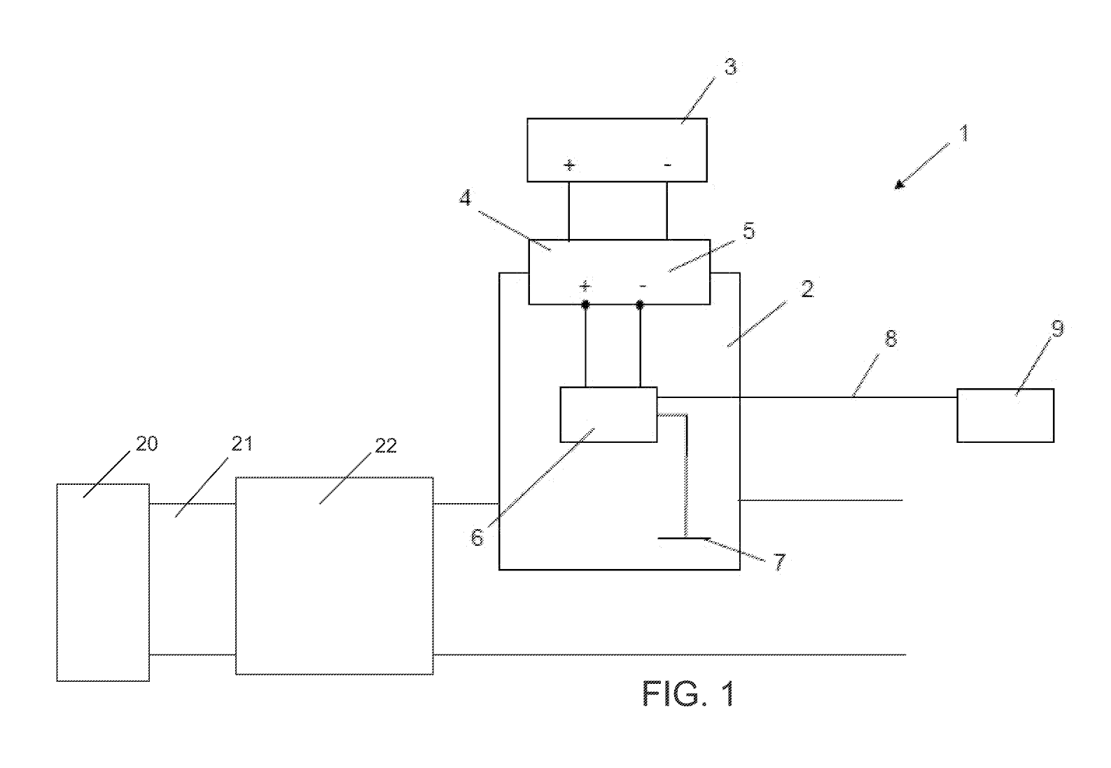

FIG. 1 schematically shows a part of an exhaust system 1 for a motor vehicle with an internal combustion engine. A particle sensor 2 detects particles or solid constituents in the exhaust-gas flow of the motor vehicle.

The motor vehicle comprises a battery system 3 which, as is conventional, provides a voltage of 12 volts. The particle sensor 2 is connected to the battery system 3, wherein the voltage of 12 volts serves as a supply or reference voltage.

The particle sensor 2 has a plug connector 4 which serves as a detachable connection to the battery system 3. The plug connector 4 is usually connected to the battery system 3 not directly but rather indirectly via one or more wiring looms, such that the plug connector 4 can be plugged into a part of a wiring loom.

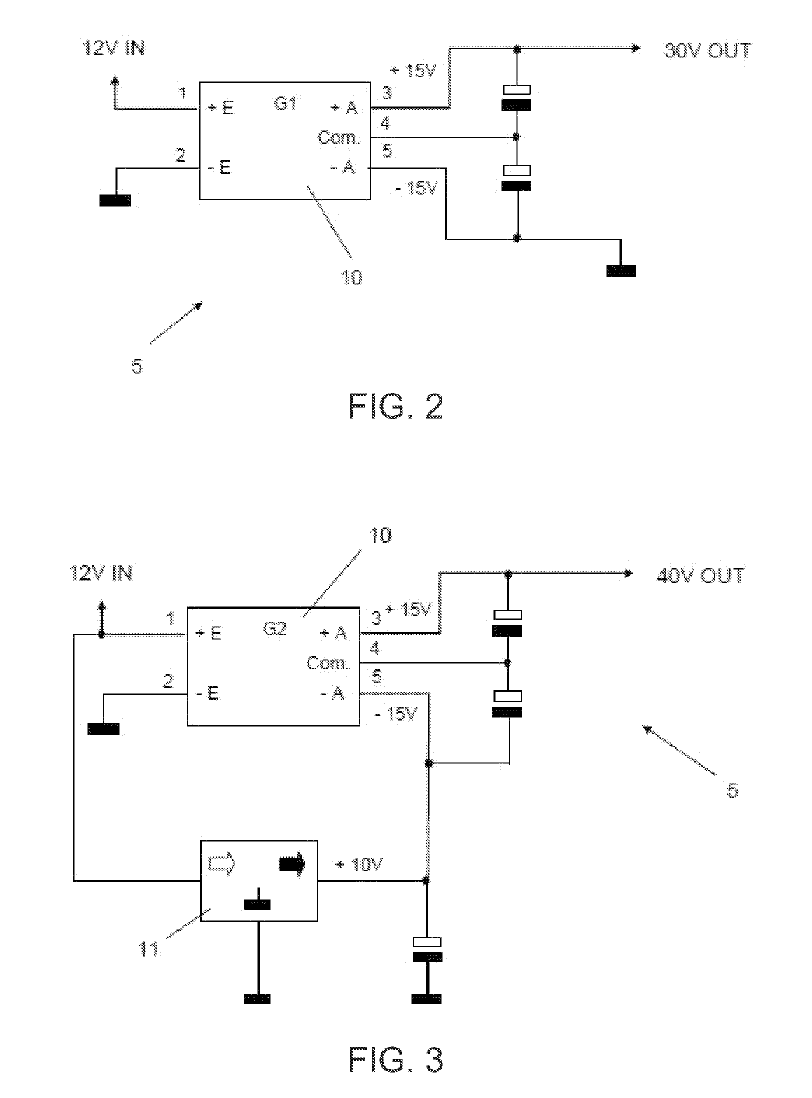

Arranged in the plug connector 4 or in the particle sensor 2 on the plug connector 4 is an amplifier circuit 5 for amplifying the externally supplied supply or reference voltage of 12 volts. The amplifier circuit 5 is integrated into the particle sensor 2, that is to say the two form a structural unit. Said unit may be of separable or non-separable design. The amplifier circuit 5 will be described in detail below on the basis of FIGS. 2 and 3.

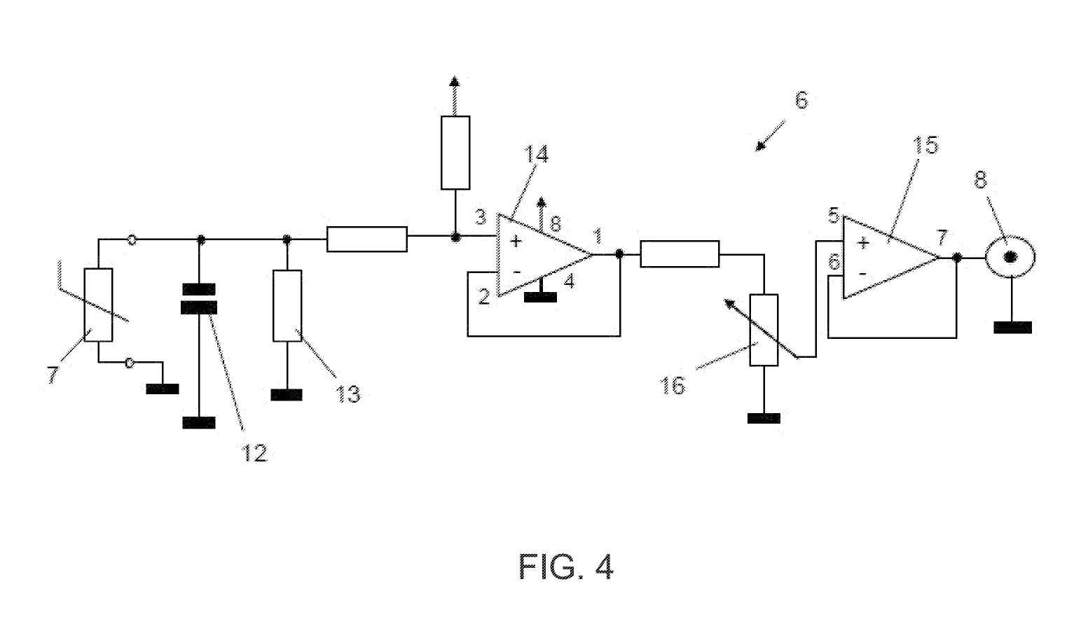

The particle sensor 2 also comprises a measurement circuit 6 which is connected to the amplifier circuit 5. The amplifier circuit 5 provides the amplified supply or reference voltage to the measurement circuit 6. The measurement circuit will be described in detail below on the basis of FIG. 4.

The particle sensor 2 comprises a sensor element 7 which is exposed to the exhaust-gas flow and whose resistance is for example measured, which resistance serves as a measure of the particles deposited on the sensor element 7. The sensor element 7 is connected to the measurement circuit 6, which evaluates or samples the sensor element 7.

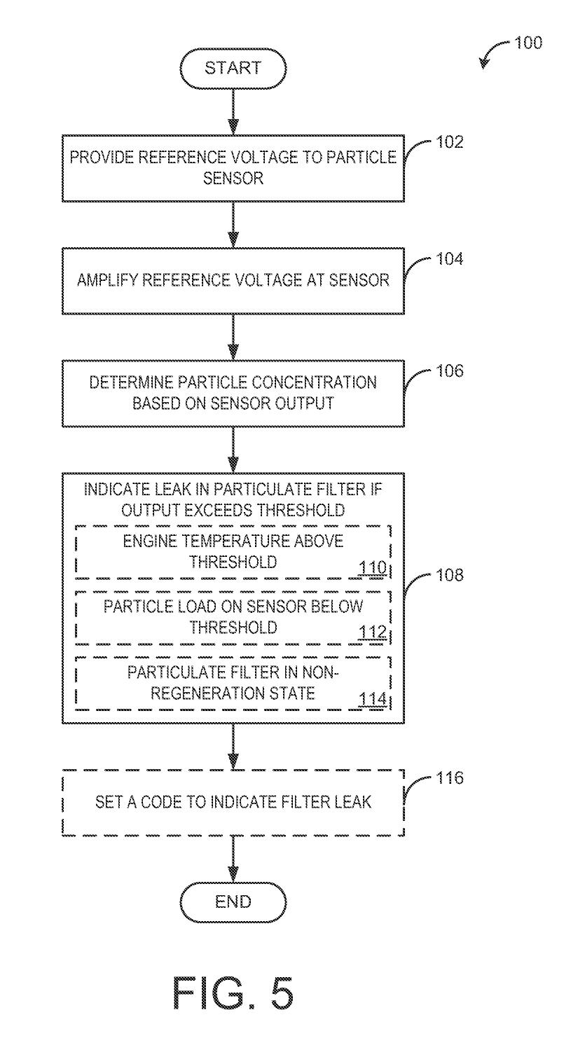

A measurement signal, usually with a voltage of 0-10 volts, is output via an output 8 of the particle sensor 2, for example to a control unit 9 of the drivetrain. The control unit may include instructions that are executable to carry out one or more control routines, such as the method described below with respect to FIG. 5.

Also shown in FIG. 1 is an engine 20 coupled to an exhaust passage 21. Positioned within the exhaust passage 21 is a particulate filter 22. The particulate filter is configured to trap particulates created in the engine 20 during combustion. These particulates may be stored until a threshold load on the filter is reached, and then the particulate filter may be regenerated by being operated at an elevated temperature. The particle sensor 2 is arranged in the exhaust passage 21 downstream of the particulate filter 22. Other elements not depicted in FIG. 1 that may also be included in the embodiments described herein include various sensors to send output to control unit 9, such as a temperature sensor to determine a temperature of the engine and/or exhaust, and a malfunction indicator light or other mechanism that receives a signal from control unit 9 in order to notify a vehicle operator or other user of a leak in the particulate filter, as will be described in more detail with respect to FIG. 5.

FIG. 2 shows a first variant of the amplifier circuit 5 which, from an input voltage of 12 volts, generates an amplified reference voltage of 30 volts.

The amplifier circuit 5 comprises a direct-current converter 10, for example of type TMR 1223, the inputs and outputs of which are potential-free. The supply voltage of the battery system 3 of 12 volts, which serves as a reference voltage for known particle sensors, is applied to the inputs of the direct-current converter 10. The direct-current converter 10 amplifies said voltage to a reference voltage of 30 volts for the particle sensor 2 proposed here. An output circuit in the form of two capacitors is situated at the outputs of the direct-current converter 10; suitable for this purpose are inter alia electrolytic capacitors composed for example of tantalum with a capacitance of 10 μF at 35 volts.

FIG. 3 shows a second variant of the amplifier circuit 5 which, from an input voltage of 12 volts, generates an amplified reference voltage of 40 volts. As in FIG. 2, a voltage converter 10 with an output circuit is used; identical or similar components may be used.

Furthermore, a voltage regulator 11 is provided which is connected in parallel with the voltage converter 10. The voltage regulator 11 generates an output voltage of 10 volts from the input voltage of 12 volts, and may be of type LM12940T 10.0. The output circuit now comprises three capacitors. The voltage converter 10 and the voltage regulator 11 are interconnected such that the summed output voltages thereof yield a reference voltage of 40 volts.

FIG. 4 illustrates, by way of example, a measurement circuit 6 for evaluating the sensor element 7. A load capacitor 12 and an input resistor 13 of the measurement circuit 6 are connected in parallel with the sensor element 7. Here, the sensor element 7 is a resistance element whose resistance varies with the number of accumulated particles.

Arranged in the further course of the circuit is an operational amplifier 14 with an input circuit in the form of two resistors. The operational amplifier 14 and the input circuit receive the amplified reference voltage of 30 or 40 volts from the amplifier circuit 5. As an operational amplifier 14, use may for example be made of the components MC33172 a, for 30 and 40 volts, or LM258 a, for 30 volts.

A further operational amplifier 15 serves as an output driver for providing the measurement signal at the output 8, which may be designed as a BNC bush. Connected upstream of the operational amplifier 15 is a regulable resistor 16 by which the amplitude of the measurement signal at the output 8 can be set in a range from 0-10 volts.

A measurement by the particle sensor 2 takes place as follows. The supply, reference or base voltage of 12 volts is supplied to the particle sensor 2 by the battery system 3. In the particle sensor 2, said reference voltage is amplified by the amplifier circuit 5 to a voltage level of 30 or 40 volts. Using said amplified reference voltage, by the measurement circuit 6 of the particle sensor 2, the sensor element 7 is measured and a measurement signal is generated. The measurement signal is provided at the output 8 of the particle sensor 2 and is transmitted to further systems, for example control units or control processors, of the motor vehicle, of the drivetrain, of the engine and/or of the exhaust-gas aftertreatment system.

Turning to FIG. 5, a method 100 for a particle sensor is presented. Method 100 may be carried out by a control system of an engine, such as control unit 9, in response to feedback from one or more sensors in the engine, such as particle sensor 2. Method 100 includes, at 102, providing a reference voltage to a particle sensor positioned in an exhaust system of the engine. The reference voltage may be received at the particle sensor from a battery, as explained above. The reference voltage may be a voltage of 12 volts or less, provided in response to an indication from the control unit, or it may be provided automatically when the engine is operated. At 104, method 100 includes amplifying the reference voltage at the particle sensor with an amplifier circuit coupled to the sensor. The amplifier circuit may amplify the reference voltage to a voltage of 30-40 volts in order to increase the sensitivity of the sensor. At 106, the particle concentration in exhaust is determined based on the output from the particle sensor, as explained above.

At 108, a leak in a particulate filter upstream of the particle sensor may be indicated if the particle concentration exceeds a threshold value. The threshold may be a suitable threshold that indicates a higher-than expected particle concentration. The threshold may be set in advance based on a determined particle concentration in the exhaust with a non-leaking particulate filter, or may be adjusted based on operating conditions, such as engine speed, load, temperature, age of the particulate filter, etc. In some embodiments, a leak may be indicated only under select operating conditions. These select operating conditions may include engine temperature above a threshold at 110. The threshold engine temperature may be a temperature at which the sensor is warm enough to provide accurate readings, or it may be another suitable temperature, such as a catalyst light-off temperature. The select operating conditions may also include a particle load on the sensor being below a threshold at 112. As explained previously, the particle sensor may accumulate particulate matter until it reaches a threshold, at which point the particle sensor may be operated at elevated temperature to remove the accumulated particulates. During this time, the sensor may not provide accurate readings. The select conditions may also include the particulate filter upstream of the particle sensor being in a non-regeneration state at 114. The particulate filter may be periodically regenerated to remove trapped particulates. Filter regeneration may be determined based on time since a previous regeneration, or other suitable mechanism.

If a leak is indicated in the particulate filter, method 100 includes setting a code to indicate the leak at 116. This may alternatively or additionally include lighting a malfunction indicator to notify a vehicle operator of the leak.

It will be appreciated that the configurations and methods disclosed herein are exemplary in nature, and that these specific embodiments are not to be considered in a limiting sense, because numerous variations are possible. For example, the above technology can be applied to V-6, I-4, I-6, V-12, opposed 4, and other engine types. The subject matter of the present disclosure includes all novel and non-obvious combinations and sub-combinations of the various systems and configurations, and other features, functions, and/or properties disclosed herein.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A particle sensor for an exhaust system, comprising:
a sensor element; and
an integrated amplifier circuit for amplifying a reference voltage for the sensor element.

2. The particle sensor as claimed in claim 1, wherein the amplifier circuit is arranged in a plug connector of the particle sensor.

3. The particle sensor as claimed in claim 1, wherein the amplifier circuit generates a reference voltage of 30-40 volts.

4. The particle sensor as claimed in claim 1, wherein the amplifier circuit has a direct-current converter.

5. The particle sensor as claimed in claim 1, wherein the amplifier circuit has a voltage regulator.

6. The particle sensor as claimed in claim 1, further comprising a measurement circuit connected to the sensor element.

7. The particle sensor as claimed in claim 6, wherein the amplifier circuit is connected to the measurement circuit in order to provide the amplified reference voltage.

8. An exhaust system for a motor vehicle with an internal combustion engine, having a particle sensor as claimed in claim 1.

9. A method for a particle sensor in the exhaust gas of an internal combustion engine, comprising:
providing a reference voltage;
amplifying the reference voltage in the particle sensor; and
determining a concentration of particles based on output from the particle sensor.

10. The method of claim 9, wherein providing a reference voltage further comprises providing a reference voltage of 12 volts from a battery of the internal combustion engine.

11. The method of claim 9, wherein amplifying the reference voltage in the particle sensor further comprises amplifying the reference voltage with an amplifier circuit to a voltage of 30-40 volts.

12. The method of claim 9, wherein the reference voltage is provided from a battery of the engine.

13. The method of claim 9, wherein determining the concentration of particles based on output from the particle sensor further comprises determining a resistance of the particle sensor.

14. An engine system, comprising:
a particulate filter positioned in an exhaust system;
a particle sensor downstream of the particulate filter, the particle sensor including an amplifying circuit to amplify a reference voltage from a battery; and
a control unit including instructions to:
under a select conditions, indicate a leak in the particulate filter if an output of the particle sensor exceeds a threshold value.

15. The engine system of claim 14, wherein the reference voltage is 12 volts.

16. The engine system of claim 14, wherein the reference voltage is amplified to 30 volts in the amplifying circuit.

17. The engine system of claim 14, wherein the reference voltage is amplified to 40 volts in the amplifying circuit.

18. The engine system of claim 14, wherein the select conditions comprise engine temperature being above a threshold.

19. The engine system of claim 14, wherein the select conditions comprise a particulate load on the particle sensor being below a threshold.

20. The engine system of claim 14, wherein the select conditions comprise the particulate filter being in a non-regeneration state.
